(19) United States
(12) Patent Application Publication
(10) Pub. No.: US 2022/0068445 A1
(43) Pub. Date: Mar. 3, 2022

(54) ROBUST FORECASTING SYSTEM ON IRREGULAR TIME SERIES IN DIALYSIS MEDICAL RECORDS

(71) Applicant: NEC Laboratories America, Inc., Princeton, NJ (US)

(72) Inventors: Jingchao Ni, Princeton, NJ (US); Bo Zong, West Windsor, NJ (US); Wei Cheng, Princeton Junction, NJ (US); Haifeng Chen, West Windsor, NJ (US); Yinjun Wu, Philadelphia, PA (US)

(21) Appl. No.: 17/408,769

(22) Filed: Aug. 23, 2021

Related U.S. Application Data
(60) Provisional application No. 63/072,325, filed on Aug. 31, 2020.

(51) Int. Cl. G16H 10/60; G06N 20/00; G06N 5/04; G16H 50/30

(52) U.S. Cl. CPC G16H 10/60; G16H 50/30; G06N 5/04; G06N 20/00

(57) ABSTRACT

A method for managing data of dialysis patients by employing a Deep Dynamic Gaussian Mixture (DDGM) model to forecast medical time series data is presented. The method includes filling missing values in an input multivariate time series by model parameters, via a pre-imputation component, by using a temporal intensity function based on Gaussian kernels and multi-dimensional correlation based on correlation parameters to be learned and storing, via a forecasting component, parameters that represent cluster centroids used by the DDGM to cluster time series for capturing correlations between different time series samples.

### RELATED APPLICATION INFORMATION

This application claims priority to Provisional Application No. 63/072,325, filed on Aug. 31, 2020, the contents of which are incorporated herein by reference in their entirety.

### BACKGROUND

### Technical Field

The present invention relates to multivariate time series analysis and, more particularly, to a robust forecasting system on irregular time series in dialysis medical records.

### Description of the Related Art

Forecasting on sparse multivariate time series (MTS) aims to model the predictors of future values of time series given their incomplete past, which is beneficial for many emerging applications. However, most existing methods process MTS's individually, and do not leverage the dynamic distributions underlying the MTS's, leading to sub-optimal results when the sparsity is high.

### SUMMARY

A method for managing data of dialysis patients by employing a Deep Dynamic Gaussian Mixture (DDGM) model to forecast medical time series data is presented. The method includes filling missing values in an input multivariate time series by model parameters, via a pre-imputation component, by using a temporal intensity function based on Gaussian kernels and multi-dimensional correlation based on correlation parameters to be learned, and storing, via a forecasting component, parameters that represent cluster centroids used by the DDGM to cluster time series for capturing correlations between different time series samples.

A non-transitory computer-readable storage medium comprising a computer-readable program for managing data of dialysis patients by employing a Deep Dynamic Gaussian Mixture (DDGM) model to forecast medical time series data is presented. The computer-readable program when executed on a computer causes the computer to perform the steps of filling missing values in an input multivariate time series by model parameters, via a pre-imputation component, by using a temporal intensity function based on Gaussian kernels and multi-dimensional correlation based on correlation parameters to be learned, and storing, via a forecasting component, parameters that represent cluster centroids used by the DDGM to cluster time series for capturing correlations between different time series samples.

A system for managing data of dialysis patients by employing a Deep Dynamic Gaussian Mixture (DDGM) model to forecast medical time series data is presented. The system includes a pre-imputation component for filling missing values in an input multivariate time series by model parameters by using a temporal intensity function based on Gaussian kernels and multi-dimensional correlation based on correlation parameters to be learned, and a forecasting component for storing parameters that represent cluster centroids used by the DDGM to cluster time series for capturing correlations between different time series samples.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block/flow diagram of an exemplary table illustrating missing values in medical time series, in accordance with embodiments of the present invention;

FIG. 2 is a block/flow diagram of an exemplary Deep Dynamic Gaussian Mixture (DDGM) architecture, in accordance with embodiments of the present invention;

FIG. 3 is a block/flow diagram of the pre-imputation component and the forecasting component of the DDGM, in accordance with embodiments of the present invention;

FIG. 4 is a block/flow diagram of an exemplary inference network of the forecasting component of the DDGM, in accordance with embodiments of the present invention;

FIG. 5 is a block/flow diagram of an exemplary generative network of the forecasting component of the DDGM, in accordance with embodiments of the present invention;

FIG. 6 is a block/flow diagram of an exemplary inverse distance weighting mechanism, in accordance with embodiments of the present invention;

FIG. 7 is a block/flow diagram of the process for employing the pre-imputation component and the forecasting component of the DDGM, in accordance with embodiments of the present invention;

FIG. 8 is an exemplary practical application for the DDGM, in accordance with embodiments of the present invention;

FIG. 9 is an exemplary processing system for the DDGM, in accordance with embodiments of the present invention; and

FIG. 10 is a block/flow diagram of an exemplary method for executing the DDGM, in accordance with embodiments of the present invention.

### DETAILED DESCRIPTION OF PREFERRED EMBODIMENTS

A generative model is introduced, which tracks the transition of latent clusters, instead of isolated feature representations, to achieve robust modeling. The generative model is characterized by a dynamic Gaussian mixture distribution, which captures the dynamics of clustering structures, and is used for providing time series. The generative model is parameterized by neural networks. A structured inference network is also implemented for enabling inductive analysis. A gating mechanism is further introduced to dynamically tune the Gaussian mixture distributions.

Multivariate time series (MTS) analysis is used in a variety of applications, such as cyber-physical system monitoring, financial forecasting, traffic analysis, and clinical diagnosis. Recent advances in deep learning have spurred on many innovative machine learning models on MTS data, which have shown remarkable results on a number of fundamental tasks, including forecasting, event prediction, and anomaly detection. Despite these successes, most existing models treat the input MTS as homogeneous and as having complete sequences. In many emerging applications, however, MTS signals are integrated from heterogeneous sources and are very sparse.

For example, MTS signals collected for dialysis patients can have several missing values. Dialysis is an important renal replacement therapy for purifying the blood of patients whose kidneys are not working normally. Dialysis patients have routines of dialysis, blood tests, chest X-ray, etc., which record data such as venous pressure, glucose level, and cardiothoracic ratio (CTR). These signal sources may have different frequencies. For instance, blood tests and CTR are often evaluated less frequently than dialysis. Different sources may not be aligned in time and what makes things worse is that some sources may be irregularly sampled, and missing entries may present. Despite such discrepancies, different signals give complementary views on a patient's physical condition, and therefore are all important to the diagnostic analysis. However, simply combining the signals will induce highly sparse MTS data. Similar scenarios are also found in other domains, e.g., in finance, time series from financial news, stock markets, and investment banks are collected at asynchronous time steps, but are strongly correlated. In large-scale complex monitoring systems, sensors of multiple sub-components may have different running environments, thus continuously producing asynchronous time series that may still be related.

The sparsity of MTS signals when integrated from heterogeneous sources presents several challenges. In particular, it complicates temporal dependencies and prevents popular models, such as recurrent neural networks (RNNs), from being directly used. The most common way to handle sparsity is to first impute missing values, and then make predictions on the imputed MTS. However, this two-step approach fails to account for the relationship between missing patterns and predictive tasks, leading to sub-optimal results when the sparsity is severe.

Recently, some end-to-end models have been proposed. One approach considers missing time steps as intervals, and designs RNNs with continuous dynamics via functional decays between observed time steps. Another approach is to parameterize all missed entries and jointly train the parameters with predictive models, so that the missing patterns are learned to fit downstream tasks. However, these methods have the drawback that MTS samples are assessed individually. Latent relational structures that are shared by different MTS samples are seldom explored for robust modeling.

In many applications, MTS's are not independent, but are related by hidden structures. In one instance, throughout the course of treatments of two dialysis patients, each patient may experience different latent states, such as kidney disorder and anemia, which are externalized by time series, such as glucose, albumin, and platelet levels. If two patients have similar pathological conditions, some of their data may be generated from similar state patterns and can form clustering structures. Thus, inferring latent states and modeling their dynamics are promising for leveraging the complementary information in clusters, which can alleviate the issue of sparsity. This concept is not limited to the medical domain. For example, in meteorology, nearby observing stations that monitor climate may experience similar weather conditions (latent states), which govern the generation of metrics, such as temperature and precipitation, over time. Although promising, inferring the latent clustering structures while modeling the dynamics underlying sparse MTS data is a challenging issue.

To address this issue, the exemplary embodiments introduce a Dynamic Gaussian Mixture based Deep Generative Model (DGM²). DGM² has a state space model under a non-linear transition emission framework. For each MTS, DGM² models the transition of latent cluster variables, instead of isolated feature representations, where all transition distributions are parameterized by neural networks. DGM² is characterized by its emission step, where a dynamic Gaussian mixture distribution is proposed to capture the dynamics of clustering structures. For inductive analysis, the exemplary embodiments resort to variational inferences, and implement structured inference networks to approximate posterior distributions. To ensure reliable inferences, the exemplary embodiments also adopt the paradigm of parametric pre-imputation and link a pre-imputation layer ahead of the inference networks. The DGM² model is designed to handle discrete variables and is constructed to be end-to-end trainable.

Thus, the exemplary embodiments investigate the issue of sparse MTS forecasting by modeling the latent dynamic clustering structures. The exemplary embodiments introduce DGM², a deep generative model that leverages the transition of latent clusters and the emission from a dynamic Gaussian mixture for robust forecasting.

As suggested by the joint imputation-prediction framework, a sparse MTS sample can be represented with missing entries against a set of evenly spaced reference time points t=1, . . . , w.

Let x_{l:w}=(xₗ, . . . , x_{w})∈ [symbol] ^{dxw} be a length-w MTS recorded from time steps l to w, where xₜ=(xₜ¹, . . . , xₜ^{d})^{T}∈ [symbol] ^{d} is a temporal feature vector at the t-th time step, xₜⁱ is the i-th variable of xₜ, and d is the total number of variables. To mark observation times, the exemplary embodiments employ a binary mask m_{1:w}=(m₁, m₂, . . . , m_{w})∈{0, 1}^{dxw}, where mₜⁱ=1 indicates xₜⁱ is an observed entry, mₜⁱ=0 otherwise, with a corresponding placeholder xₜⁱ=NaN.

The exemplary embodiments are focused on a sparse MTS forecasting problem, which estimates the most likely length-r sequence in the future given the incomplete observations in past w time steps, e.g., the exemplary embodiments aim to obtain:

$${\tilde{x}}_{ω + 1 : ω + r} = {arg max}_{{x}_{ω + 1 : ω + r}} p ( {x}_{ω + 1 : ω + r} \vert {x}_{1 : ω} , {m}_{1 : ω} )$$

where \tilde{x}_{w+1:w+r}=(\tilde{x}_{w+1}, . . . , \tilde{x}_{w+r}) are predicted estimates and p(⋅|⋅) is a forecasting function to be learned.

The exemplary embodiments introduce the DGM² model as follows. Inspired by the successful paradigm of joint imputation and prediction, the exemplary embodiments design DGM² to have a pre-imputation layer for capturing the temporal intensity and the multi-dimensional correlations present in every MTS, for parameterizing missing entries. The parameterized MTS is fed to a forecasting component, which has a deep generative model that estimates the latent dynamic distributions for robust forecasting.

Regarding the pre-imputation layer, this layer aims to estimate the missing entries by leveraging the smooth trends and temporal intensities of the observed parts, which can help alleviate the impacts of sparsity in the downstream predictive tasks.

For the i-th variable at the t*-th reference time point, the exemplary embodiments use a Gaussian kernel k(t*, t; αᵢ)=e^{-α}^{ᵢ}^{(t*-t)}^{²} to evaluate the temporal influence of any time step t (1≤t≤w) on t*, where αᵢ is a parameter to be learned. Based on the kernel, the exemplary embodiments then employ a weighted aggregation for estimating x_{t*}ⁱ by:

$${\overline{x}}_{{t}^{*}}^{2} = \frac{1}{λ ( {t}^{*} , {m}^{i} ; {α}_{i} )} {\sum }_{i = 1}^{ω} κ ( {t}^{*} , t ; {α}_{i} ) {m}_{t}^{i} {x}_{t}^{i}$$

where mⁱ=(m₁ⁱ . . . , m_{w}ⁱ)^{T}∈ [symbol] ^{w} is the mask of the ith variable, and λ(t*; mⁱ; αᵢ)=Σₜ₌₁^{w}mₜⁱκ(t*,t;αᵢ) is an intensity function that evaluates the observation density at t*, in which mₜⁱ is used to zero out unobserved time steps.

To account for the correlations of different variables, the exemplary embodiments also merge the information across d variables by introducing learnable correlation coefficients ρᵢⱼ for i, j=1, . . . , d, and formulating a parameterized output if x_{t*}ⁱ is unobserved, such that:

$${\hat{x}}_{{i}^{*}}^{i} = [ {\sum }_{j = 1}^{d} {ρ}_{ij} λ ( {t}^{*} , {m}^{i} ; {α}_{j} ) {x}_{{t}^{*}}^{- j} ] / {\sum }_{{j}^{′} = 1}^{d} λ ( {t}^{*} , {m}^{i} ; {α}_{{j}^{′}} )$$

where ρᵢⱼ is set as 1 for i=j, and λ(t*; mⁱ; αⱼ) is introduced to indicate the reliability of x̅_{t*}^{j}, because larger λ(t*; mⁱ; αⱼ) implies more observations near x̅_{t*}^{j}.

In this layer, the set of parameters are α=[α₁, . . . , α_{d}], and ρ=[ρᵢⱼ]_{i,j=1}^{d}. DGM² trains them jointly with its generative model for aligning missing patterns with the forecasting tasks.

Regarding the forecasting component, the exemplary embodiments implement a generative model that captures the latent dynamic clustering structures for robust forecasting. Suppose there are k latent clusters underlying all temporal features xₜ's in a batch of MTS samples. For every time step t, the exemplary embodiments associate xₜ with a latent cluster variable zₜ to indicate to which cluster xₜ belongs. Instead of the transition of xₜ→xₜ₊₁, the exemplary embodiments model the transition of the cluster variables zₜ→zₜ₊₁. Since the clusters integrate the complementary information of similar features across MTS samples at different time steps, leveraging them is more robust than using individual sparse feature xₜ's.

Regarding the generative model, the generative process of the DGM² follows the transition and emission framework of state space models.

First, the transition process of DGM² employs a recurrent structure due to its effectiveness on modeling long-term temporal dependencies of sequential variables. Each time, the probability of a new state zₜ₊₁ is updated upon its previous states z_{1:t}=(z₁, . . . , zₜ). The exemplary embodiments use a learnable function to define the transition probability, e.g., p(zₜ₊₁|z_{1:t})=ƒ_{θ}(z_{1:t}) where the function ƒ_{θ}(⋅) is parameterized by θ, which can be variants of RNNs, for encoding non-linear dynamics that may be established between the latent variables.

For the emission process, the exemplary embodiments implement a dynamic Gaussian mixture distribution, which is defined by dynamically tuning a static basis mixture distribution. Let μᵢ(i=1, . . . , k) be the mean of the i-th mixture component of the basis distribution, and p(μᵢ) be its corresponding mixture probability. The emission (or forecasting) of a new feature xₜ₊₁ at time step t+1 involves the following steps, that is, drawing a latent cluster variable zₜ₊₁ from a categorical distribution on all mixture components and drawing xₜ₊₁ from the Gaussian distribution N(μ_{z}_{ₜ₊₁}, σ⁻¹l), where σ is a hyperparameter, and I is an identity matrix. The exemplary embodiments use isotropic Gaussian because of its efficiency and effectiveness.

In the first step, the categorical distribution is usually defined on p(μ)=[p(μ₁), . . . , p(μₖ)] ∈ [symbol] ^{k}, e.g., the static mixture probabilities, which cannot reflect the dynamics in MTS. In light of this, and considering the fact that transition probability p(zₜ₊₁|z_{1:t}) indicates to which cluster xₜ₊₁ belongs, the exemplary embodiments dynamically adjust the mixture probability at each time step using p(zₜ₊₁|z_{1:t}) by:

$${ψ}_{t + 1} = \underbrace{( 1 - γ ) p ( {z}_{t + 1} \vert {z}_{1 : t} )}_{dynamic adjustment} + \underbrace{γ p ( μ )}_{basis mixture}$$

where ψₜ₊₁ is the dynamic mixture distribution at time step t+1, and γ is a hyperparameter within [0, 1] that controls the relative degree of change that deviates from the basis mixture distribution.

The dynamic adjustment process of ψₜ₊₁ on a Gaussian mixture with two components can be shown where p(zₜ₊₁|z_{1:t}) adjusts the mixture towards the component (e.g., cluster) that xₜ₊₁ belongs to. It is noteworthy that adding the basis mixture in ψₜ₊₁ is beneficial because it determines the relationships between different components, which regularizes the learning of the means μ=[μ₁, . . . , μₖ] during model training.

As such, the generative process can be summarized for each MTS sample:

(a) draw z₁˜Uniform(k)

(b) for time step t=1, . . . , w:

i. compute the transition probability by: p(zₜ₊₁|z_{1:t})=ƒ_{θ}(z_{1:t})

ii. draw zₜ₊₁˜Categorial p (zₜ₊₁|z_{1:t}) for transition.

iii. draw \tilde{z}ₜ₊₁˜Categorial ψₜ₊₁ using ψₜ₊₁ for emission

iv. draw a feature vector \tilde{x}ₜ₊₁˜ [symbol] (μ_{\tilde{z}}_{ₜ₊₁},σ⁻¹I)

where zₜ₊₁ (step ii) and \tilde{z}ₜ₊₁ (step iii) are different. zₜ₊₁ is used in transition (step i) for maintaining recurrent property and \tilde{z}ₜ₊₁ is used in emission from updated mixture distribution.

In the above process, the parameters in p are shared by samples in the same cluster, whereby consolidating complementary information for robust forecasting.

Regarding parameterization of the generative model, the parametric function in the generative process is ƒ_{θ}(⋅), for which the exemplary embodiments choose a recurrent neural network architecture as:

p(zₜ₊₁|z_{1:t})=softmax(MLP(hₜ))

where hₜ=RNN(zₜ,hₜ₋₁)

and hₜ is the t-th hidden state, MLP represents a multilayer perceptron, RNN can be instantiated by either a long short-term memory (LSTM) or a gated recurrent network (GRU). Moreover, to accommodate the applications where the reference time steps of MTS's could be unevenly spaced, the exemplary embodiments can also incorporate the neural ordinary differential equations (ODE) based RNNs to handle the time intervals.

In summary, the set of trainable parameters of the generative model is ϑ={θ, μ}. Given this, the exemplary embodiments aim at maximizing the log marginal likelihood of observing each MTS sample, e.g.,

$$ℒ ( ϑ ) = log ( {\sum }_{{z}_{1 : ω}} {p}_{ϑ} ( {x}_{1 : ω} , {z}_{1 : ω} ) )$$

where the joint probability in [symbol] (ϑ) can be factorized with respect to the dynamic mixture distribution in ψₜ₊₁ after the Jensen's inequality is applied on [symbol] (ϑ) by:

$$ℒ ( ϑ ) \geq {\sum }_{t = 0}^{ω - 1} {\sum }_{{z}_{1 : t + 1}} [ log ( {p}_{θ} ( {x}_{t + 1} \vert {z}_{t + 1} ) ) {p}_{θ} ( {z}_{1 : t} ) [ ( 1 - γ ) {p}_{θ} ( {z}_{t + 1} \vert {z}_{1 : t} ) + γ p ( {μ}_{{z}_{t + 1}} ) ] ]$$

in which the above lower bound will serve as the objective to be maximized.

In order to estimate the parameters ϑ, the goal is to maximize the above equation. However, summing out z_{1:t+1} over all possible sequences is computationally difficult. Therefore, evaluating the true posterior density p(z|x_{1:w}) is intractable. To circumvent this issue, meanwhile enabling inductive analysis, the exemplary embodiments resort to variational inference and introduce an inference network.

Regarding the inference network, the exemplary embodiments introduce an approximated posterior q_{ϕ}(z|x_{1:w}), which is parameterized by neural networks with parameter ϕ. The exemplary embodiments design the inference network to be structural and employ deep Markov processes to maintain the temporal dependencies between latent variables, which leads to the following factorization:

$${q}_{ϕ} ( z \vert {x}_{1 : ω} ) = {q}_{ϕ} ( {z}_{1} \vert {x}_{1} ) {\prod }_{t = 1}^{ω - 1} {q}_{ϕ} ( {z}_{t + 1} \vert {x}_{1 : t + 1} , {z}_{t} )$$

With the introduction of q_{ϕ}(z|x_{1:w}), instead of maximizing the log marginal likelihood [symbol] (ϑ), the exemplary embodiments are interested in maximizing the variational evidence lower bound (ELBO) [symbol] (ϑ,φ)≤ [symbol] (ϑ) with respect to both ϑ and ϕ.

By incorporating the bounding step in [symbol] (ϑ), the exemplary embodiments can derive the EBLO of the problem, which is written by:

$$ℓ ( ϑ , ϕ ) = ( 1 - γ ) {\sum }_{t = 1} {𝔼}_{{q}_{ϕ} ( {z}_{t} \vert {x}_{1 : t} )} [ log ( {p}_{θ} ( {x}_{t} \vert {z}_{t} ) ) ] - {\sum }_{t = 1}^{ω - 1} {𝔼}_{{q}_{ϕ} ( {z}_{1 : t} \vert {x}_{1 : t} )} [ {𝒟}_{KL} ( {q}_{ϕ} ( {z}_{t + 1} \vert {x}_{1 : t + 1} , {z}_{t} ) ‖ {p}_{θ} ( {z}_{t + 1} \vert {z}_{1 : t} ) ) ] - {𝒟}_{KL} ( {q}_{ϕ} ( {z}_{1} \vert {x}_{1} ) ‖ {p}_{ϑ} ( {z}_{1} ) ) + γ {\sum }_{t = 1}^{ω} {\sum }_{{z}_{t} = 1}^{k} {p}_{θ} ( {μ}_{{z}_{t}} ) log ( {p}_{ϑ} ( {x}_{t} \vert {z}_{t} ) )$$

where [symbol] _{KL}(⋅∥⋅) is the KL-divergence and p_{ϑ}(z₁) is a uniform prior as described in the generative process. Similar to a variational autoencoder (VAE), it helps prevent overfitting and improve the generalization capability of the model.

The [symbol] (ϑ, φ) equation also sheds some insights on how the dynamic mixture distribution in ψₜ₊₁ works. For instance, the first three terms encapsulate the learning criteria for dynamic adjustments and the last term after γ regularizes the relationships between different basis mixture components.

In the architecture of the inference network, q_{ϕ}(zₜ₊₁|x_{1:t+1}, zₜ), is a recurrent structure:

q_{ϕ}(zₜ₊₁|x_{1:t+1},zₜ)=softmax(MLP(\tilde{h}ₜ₊₁))

where \tilde{h}ₜ₊₁=RNN(xₜ,\tilde{h}ₜ)

\tilde{h}ₜ is the t-th latent state of the RNNs, and z₀ is set to 0 so that it has no impact in the iteration.

Since sampling discrete variable zₜ from the categorical distributions is not differentiable, it is difficult to optimize the model parameters. To get rid of it, the exemplary embodiments employ the Gumbel-softmax reparameterization trick to generate differentiable discrete samples. In this way, the DGM² model is end-to-end trainable.

Regarding gated dynamic distributions, in ψₜ₊₁, the dynamics of the Gaussian mixture distribution are tuned by a hyperparameter γ, which may require some tuning efforts on validation datasets. To circumvent it, the exemplary embodiments introduce a gate function γ(\tilde{h}ₜ)=sigmoid (MLP(\tilde{h}ₜ)) using the information extracted by the inference network to substitute γ in ψₜ₊₁. As such, ψₜ becomes a gated distribution that can be dynamically tuned at each time step.

Regarding model training, the exemplary embodiments jointly learn the parameters {α, [symbol] , ϑ, ϕ)} of the preimputation layer, the generative network p_{ϑ}, and the inference network q_{ϕ} by maximizing the ELBO in the equation for [symbol] (ϑ,φ).

The main challenge in evaluating [symbol] (ϑ,φ) is to obtain the gradients of all terms under the expectation [symbol] _{q}_{_{ϕ}}. Because zₜ is categorical, the first term can be analytically calculated with the probability q_{ϕ}(zₜ|x_{1:t}). However, q_{ϕ}(zₜ|x_{1:t}) is not an output of the inference network, so the exemplary embodiments derive a subroutine to compute q_{ϕ}(zₜ|x_{1:t}) from q_{ϕ}(zₜ|x_{1:t}, zₜ₋₁). In the second term, since the KL divergence is sequentially evaluated, the exemplary embodiments employ ancestral sampling techniques to sample zₜ from time step l to w to approximate the distribution q_{ϕ}. It is also noteworthy that in [symbol] (ε, φ), the exemplary embodiments only evaluate observed values in xₜ by using masks mₜ to mask out the unobserved parts.

As such, the entire DGM² is differentiable, and the exemplary embodiments use stochastic gradient descents to optimize [symbol] (ε, φ). In the last term of the equation for [symbol] (ε, φ), the exemplary embodiments also need to perform density estimation of the basis mixture distribution, e.g., to estimate p(μ).

Given a batch of MTS samples, suppose there are n temporal features xₜ in this batch, and their collection is denoted by a set X, the exemplary embodiments can then estimate the mixture probability by:

$$p ( {μ}_{i} ) = {\sum }_{{x}_{t} ∈ X} {q}_{ϕ} ( {z}_{t} = i \vert {x}_{1 : t} , {z}_{t - 1} ) / n , for i = 1 , … , k$$

where q_{ϕ}(zₜ=i|x_{1:t}, zₜ₋₁) is the inferred membership probability of xₜ to the i-th latent cluster by q_{ϕ}(zₜ₊₁|x_{1:t+1}, zₜ)=softmax(MLP(\tilde{h}ₜ₊₁)).

Moving back to time series forecasting in the medical domain, the tremendous employments of digital systems in hospitals and many medical institutions have brought forth a large volume of healthcare data of patients. The big data are of substantial value, which enables artificial intelligence (AI) to be exploited to support clinical judgement in medicine. As one of the critical themes in modern medicine, the number of patients with kidney diseases has raised social, medical and socioeconomic issues worldwide. Hemodialysis, or simply dialysis, is a process of purifying the blood of a patient whose kidneys are not working normally and is one of the important renal replacement therapies (RRT). However, dialysis patients at high risk of cardiovascular and other diseases require intensive management on blood pressure, anemia, mineral metabolism, and so on. Otherwise, patients may encounter critical events, such as low blood pressure, leg cramp, and even mortality, during dialysis. Therefore, medical staff must decide to start dialysis from various viewpoints.

Given the availability of big medical data, it is important to develop AI systems for making prognostic prediction of some critical medical indicators, such as blood pressure, amount of dehydration, hydraulic pressure, etc., during the pre-dialysis period. This is a time series forecasting problem in the medical domain. The major challenge of this issue is the large number of missing values present in medical records, which can account for 50%˜80% entries in the data. This is mainly because of the irregular dates on different tests for each patient.

Dialysis measurement records have a frequency of 3 times/week (e.g., blood pressure, weight, venous pressure, etc.), blood test measurements have a frequency of 2 times/month (e.g., albumin, glucose, platelet count, etc.), and cardiothoracic ratio (CTR) have a frequency of 1 time/month. The three parts are dynamic and change over time, so they can be modeled by time series, but with different frequencies.

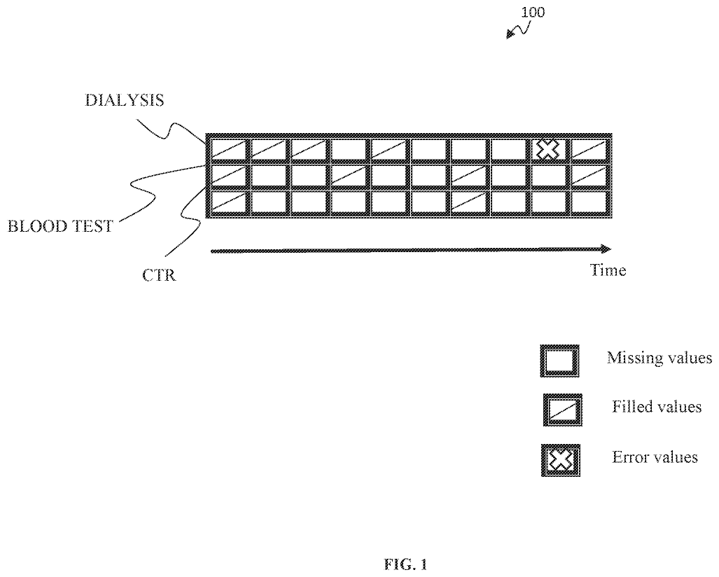

When combining these different parts of data together, low-frequency time series (e.g., blood test measurements) will have many missing entries on the dates when only high-frequency time series is recorded (e.g., dialysis measurements), as depicted in the table 100 in FIG. 1.

Also, on each testing date, there could be missing items due to not knowing, time limitations, and costs. Therefore, precise time series forecasting with presence of missing values is important for assisting the decision-making processes of medical staffs, and hence is beneficial to reduce the risk of events during dialysis.

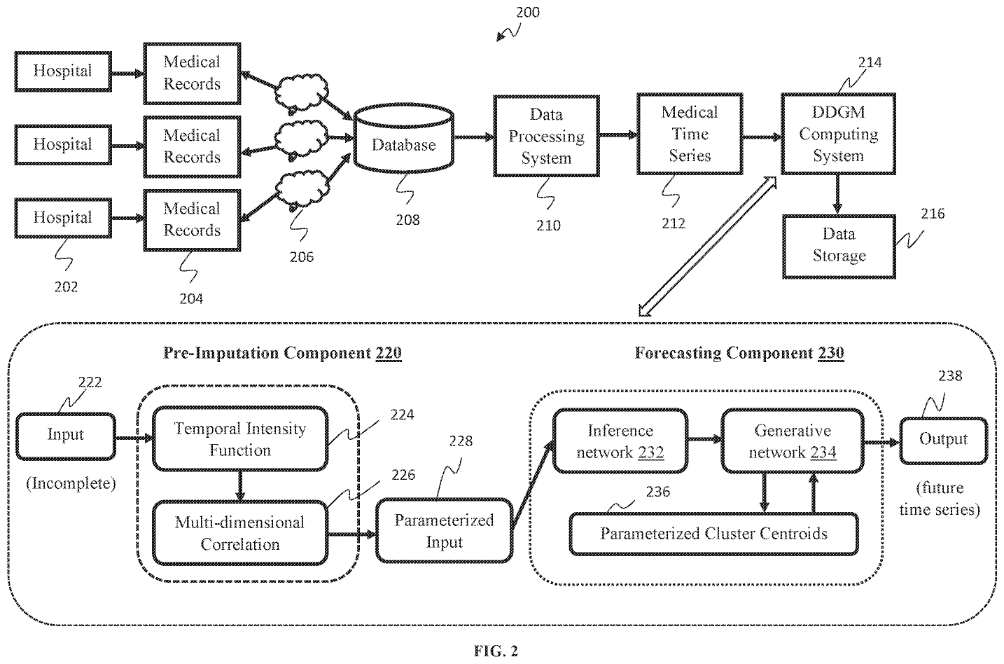

The exemplary embodiments seek to harness the potential of the management data of dialysis patients in providing automatic and high-quality forecasting of medical time series. The present invention is an artificial intelligent system. Its core computation system employs a Deep Dynamic Gaussian Mixture (DDGM) model, which enables joint imputation and forecasting of medical time series with the presence of missing values. Therefore, the system can be referred to as a DDGM system. The architecture of the DDGM system 200 is illustrated in FIG. 2.

It is also worth to mention that the DDGM system 200 is general and can be applied to other medical records data with similar format as illustrated in FIG. 1.

DDGM system 200 can include medical records 204 obtained from hospitals 202, the medical records 204 provided through clouds 206 to a database 208. A data processing system 210 processes data from the database 208 to obtain medical time series 212 to be supplied to the DDGM computing system 214. Data storage 216 can also be provided.

The DDGM computing system 214 can include a pre-computation component 220 and a forecasting component 230.

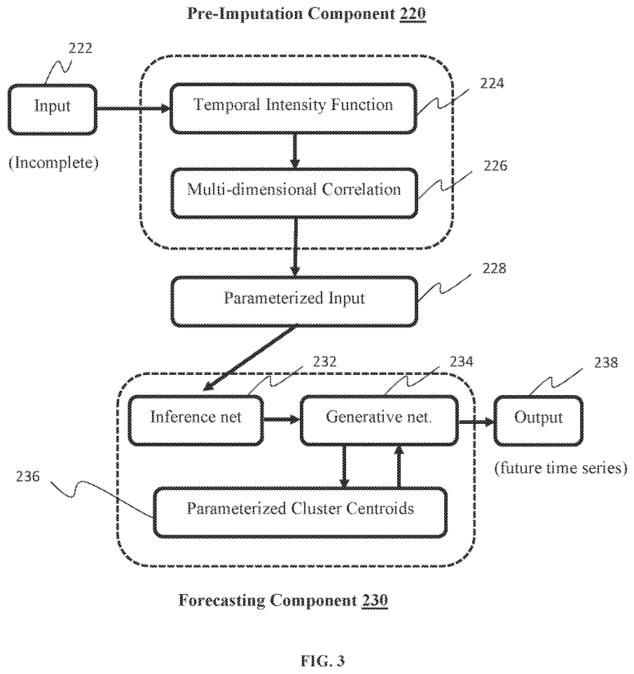

FIG. 3 shows the overall architecture of the DDGM system 200.

Regarding pre-imputation component 220, the goal of the pre-imputation component 220 is to fill missing values in the input time series by some parameterized functions, so that the parameters can be trained jointly with the forecasting tasks. After these parameters are well trained, by passing new input time series through component 220, the missing values of the time series will be automatically filled by the functions. The filled values will approximate the true measurements, and the completed output will be fed to the forecasting component 230, which facilitates reliable processing.

The pre-computation component 220 includes a temporal intensity function 224 and multi-dimensional correlation 226.

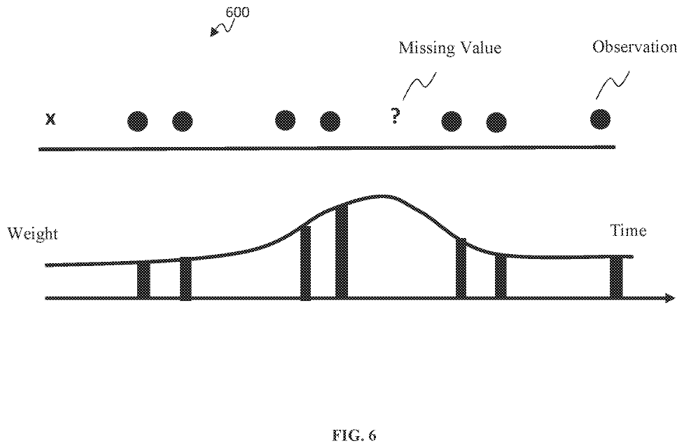

Regarding the temporal intensity function 224, this function is designed to model the temporal relationship between time steps. Missing values may depend on all the existing observations, which can be interpolated by summing up the observed values with different weights. Intuitively, the time step at which the missing value appears is mostly impacted by its closest time steps. To reflect this fact, the exemplary embodiments design the temporal intensity function 224 based on an inverse distance weighting mechanism, e.g., nearby time steps receive higher weights than faraway time steps, as illustrated in FIG. 6.

Suppose the missing value occurs at time step t* for the i-th dimension of the input multivariate time series, then the exemplary embodiments design the intensity function based on a Gaussian kernel as follows:

ƒ=Σₜ₌₁^{T}e^{-α(t-t*)}^{²}

where T is the length of the time series, and α is a parameter to learn. The relationship 600 between the output of this function and time steps is illustrated in FIG. 6.

Regarding multi-dimensional correlation, module 226 is designed to capture the correlation between different dimensions of the input multivariate time series. Suppose the time series have D dimensions in total, then module 226 initializes a matrix parameter ρΣ [symbol] ^{D×D}, which is a D by D continuous matrix. Each entry ρᵢⱼ represents the correlation between dimension i and j. This parameter matrix will also be trained with other parts of the model on the training data.

By plugging this parameter into the temporal intensity function 224, the exemplary embodiments can obtain the function that runs within the pre-imputation component 220 as:

\hat{x}_{it*}=Σⱼ₌₁^{D}Σₜ₌₁^{T}e^{-α(t-t*)}^{²}ρᵢⱼxⱼₜ

where \hat{x}_{it*} represents the imputed value of the i-th dimension at the t*-th time step. xⱼₜ is the observation of the j-th dimension at the t-th time step. The outputted \hat{x}_{it*} value will be used to fill missing values in the input time series and will be sent to the next forecasting component for processing.

Regarding the forecasting component 230, this component links the output 228 of component 220 and the downstream forecasting task. The goal of the component 230 is to learn some cluster centroids via a dynamic Gaussian mixture model for further enhancing the robustness of forecasting results. Component 230 has the capability to generate values for future time steps, for the purpose of time series forecasting.

There are, e.g., three modules or elements within component 230.

Regarding the inference network 232, the input to this module is the output 228 of component 220, that is, time series with filled missing values.

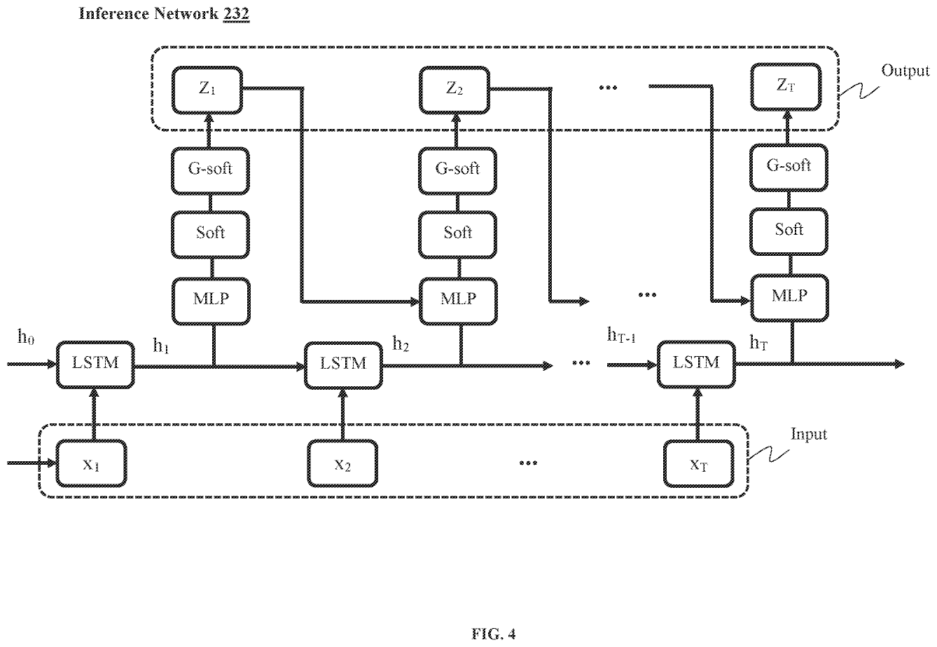

As shown in FIG. 4, suppose the time series are x₁, x₂, . . . , x_{T}, each of them will be iterative processed by a LSTM unit and output latent feature vectors h₁, h₂, . . . , h_{T} consecutively, such that hₜ=LSTM(xₜ,hₜ₋₁).

Each time a hₜ is generated, it will be sent to a sub-module with three layers, that is, MLP, softmax, and Gumbel softmax. The output of this sub-module will be a sequence of sparse vectors z₁, z₂, . . . , z_{T}, which represent the inferred cluster variable for each time step. For example, if there are k possible clusters in the data, then zₜ is a length-k vector, with the highest value indicating the cluster membership of the feature vector xₜ, such that:

zₜ=G_Softmax(Softmax(MLP(hₜ)))

The design of the inference network follows the variational inference process of the statistical model. The output vectors z₁, z₂, . . . , z_{T} are latent variables that will be used by the generative network 234 for generating/forecasting new values.

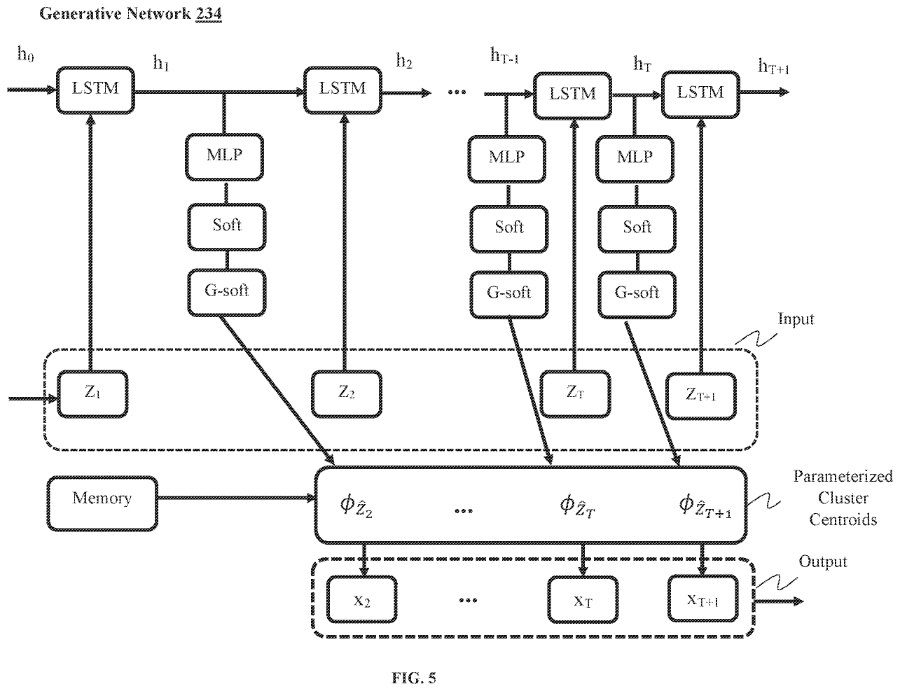

Regarding the generative network 234 and parameterized cluster centroids 236, the input to module 234 is the output of the inference network 232, e.g., latent variables z₁, z₂, . . . , z_{T}. As illustrated in FIG. 5, these variables will be iteratively processed by an LSTM unit and new latent feature vectors h₁, h₂, . . . , h_{T} are output consecutively, such as hₜ=LSTM(zₜ,hₜ₋₁).

Each time a hₜ is generated, it will be sent to another sub-module with three layers, that is, MLP, softmax, and Gumbel softmax. The output of this sub-module will be a new sequence of sparse vectors \tilde{z}₁, \tilde{z}₂, . . . , \tilde{z}_{T}, which represent the generative cluster variable for each time step.

These variables are different from those in the output of the inference network 232. This is because the output of the inference network 232 can only be up to time step T. In contrast, the output of the generative network 234 can be up to any time step after T for forecasting purposes.

Then, \tilde{z}₁, \tilde{z}₂, . . . , \tilde{z}_{T} will be sent to cluster centroid module 236 for generating a mean value vector ϕ_{\hat{z}}ₜ for t=1, . . . T. Also, t can be larger than T. Each mean value vector ϕ_{\hat{z}}ₜ is used for generating a particular measurement at time step t by drawing from a Gaussian mixture model.

That is: \tilde{z}ₜ˜Categorical(Pr(\hat{z}ₜ)),

where \hat{x}ₜ˜N(ϕ_{\hat{z}}ₜ,σ⁻¹I).

“Categorical” represents categorical distribution, N represents a Gaussian distribution, σ represents variance, and I represents an identity matrix.

In this manner, the exemplary embodiments can iteratively draw \hat{x}ₜ₊₁, \hat{x}ₜ₊₂, . . . , \hat{x}_{t+w} for forecasting future measurements for w time steps.

Regarding model training, to train the model, the exemplary embodiments maximize the likelihood on the observed training data.

The objective function to be maximized is given as:

$$\begin{matrix}L ( x \vert ϕ , θ , Ω ) = {\sum }_{t = 2}^{T} {𝔼}_{q ( {z}_{t} \vert {x}_{1 : T} )} ( log p ( {x}_{t} \vert {z}_{t} ; ϕ ) ) \\ - {\sum }_{t = 2}^{T} {𝔼}_{q ( {z}_{1 : t - 1} \vert {x}_{1 : T} )} ( {D}_{KL} ( q ( {z}_{t} \vert {z}_{t - 1} , {x}_{1 : T} ; Ω ) ‖ p ( {z}_{t} \vert {z}_{1 : t - 1} , θ ) ) ) \\ - {D}_{KL} ( q ( {z}_{1} \vert {z}_{0} , {x}_{1 : T} ; Ω ) ‖ p ( {z}_{1} ) )\end{matrix}$$

where [symbol] represents an expectation and D_{KL} represents a KL divergence function. The input to this function includes z₁, z₂, . . . , z_{T}, \tilde{z}₁, \tilde{z}₂, . . . , \tilde{z}_{T}, x₁, x₂, . . . , x_{T}, and \hat{x}₁, \hat{x}₂, . . . , \hat{x}_{T}, and the output is a value that represents the likelihood of observing the training data given the probability computations made by the DDGM 200. By maximizing this likelihood by a gradient descent algorithm, the model parameters will be trained. After the model is well trained, it can be used to perform forecasting on newly input time series.

Therefore, the methods of the exemplary embodiments can be implemented by:

Inputting the time series (with missing values) to the pre-imputation component 220.

The pre-imputation component 220 uses intensity functions and correlation parameters to fill missing values.

The output of pre-imputation component 220 is sent to the input port of the forecasting component 230.

The input of component 230 will first go through the inference network 232 to infer latent variables for time steps 1, . . . , T.

The inferred latent variables will be sent to the generative network 234 to generate another copy of cluster variables for time steps 1, . . . T.

After time step T, the generative network 234 can use its generated cluster variables as its own input to iteratively generate new cluster variables for time steps after T.

For the output of the previous steps, e.g., the generated cluster variables, they are sent to parameterize cluster centroids 236 to generate mean value vectors.

From the Gaussian mixture distribution, using the mean value vectors generated to draw forecasted measurement values at each forecasted time step.

For the training phase only, send the generated values and the observations (for t=1, . . . , T) in the training data to the objective function for model training.

In summary, the exemplary embodiments provide a systematic and big data driven solution to the problem of dialysis medical time series forecasting. The new aspects of the DDGM system lie in its computing system, which is designed to handle the missing value problem in dialysis medical time series data. A pre-imputation component is presented that fills missing values by parameterized functions (parameters are learned jointly with forecasting tasks). The pre-imputation component has a temporal intensity function, which captures temporal dependency between timestamps and multi-dimensional correlation, which captures correlation between multiple dimensions. A clustering-based forecasting component captures the correlation between different time series samples for further refining imputed values.

The advantages of the DDGM system are at least providing a three-level perspective for robust imputation, including, temporal dependency, cross-dimensional correlation, and cross-sample correlation (via clustering). Regarding joint imputation and forecasting, capturing dependencies between missing patterns and forecasting tasks is beneficial. Thus, the DDGM system is a specifically designed intelligent system that advances the state-of-the-art by the aforementioned advantages, that is, three-level robust imputation and joint imputation and forecasting.

The inventive features include at least the pre-imputation component for filling missing values by model parameters using two kinds of functions, a temporal intensity function based on Gaussian kernels and multi-dimensional correlation based on correlation parameters to be learned. The forecasting component is a generative model designed upon Gaussian mixture distribution for storing parameters that represent cluster centers, which are used by the model to cluster time series for capturing the correlations between samples. Additionally, a joint imputation and forecasting training algorithm is introduced to facilitate learning imputed values that are aligned well to the forecasting tasks.

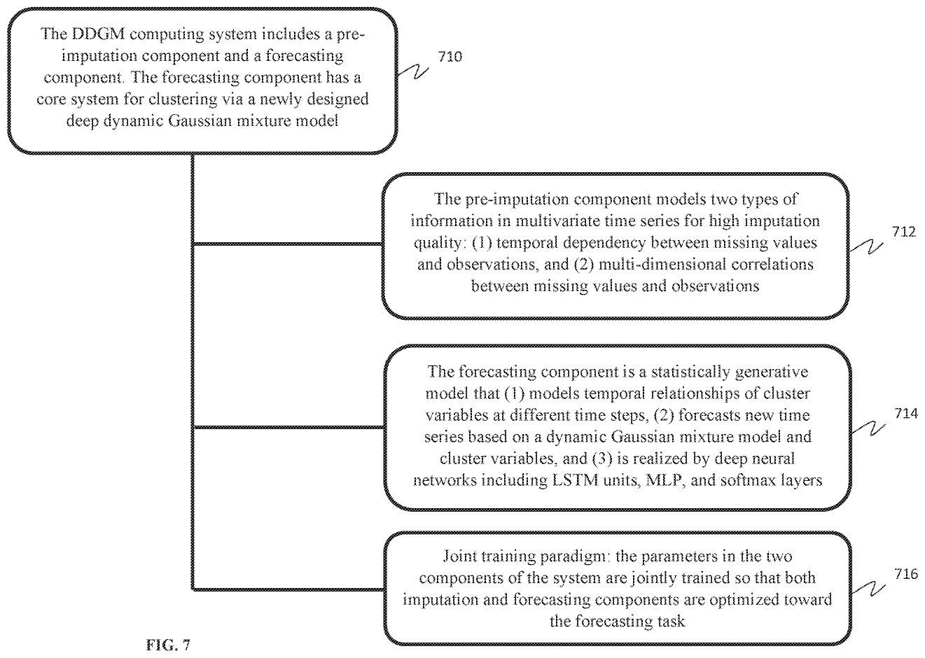

FIG. 7 is a block/flow diagram of the process for employing the pre-imputation component and the forecasting component of the DDGM, in accordance with embodiments of the present invention.

At block 710, the DDGM computing system includes a pre-imputation component and a forecasting component. The forecasting component has a core system for clustering via a newly designed deep dynamic Gaussian mixture model.

At block 712, the pre-imputation component models two types of information in multivariate time series for high imputation quality, that is, temporal dependency between missing values and observations, and multi-dimensional correlations between missing values and observations

At block 714, the forecasting component is a statistically generative model that models temporal relationships of cluster variables at different time steps, forecasts new time series based on a dynamic Gaussian mixture model and cluster variables, and is realized by deep neural networks including LSTM units, MLP, and softmax layers.

At block 716, regarding the joint training paradigm, the parameters in the two components of the system are jointly trained so that both imputation and forecasting components are optimized toward the forecasting task.

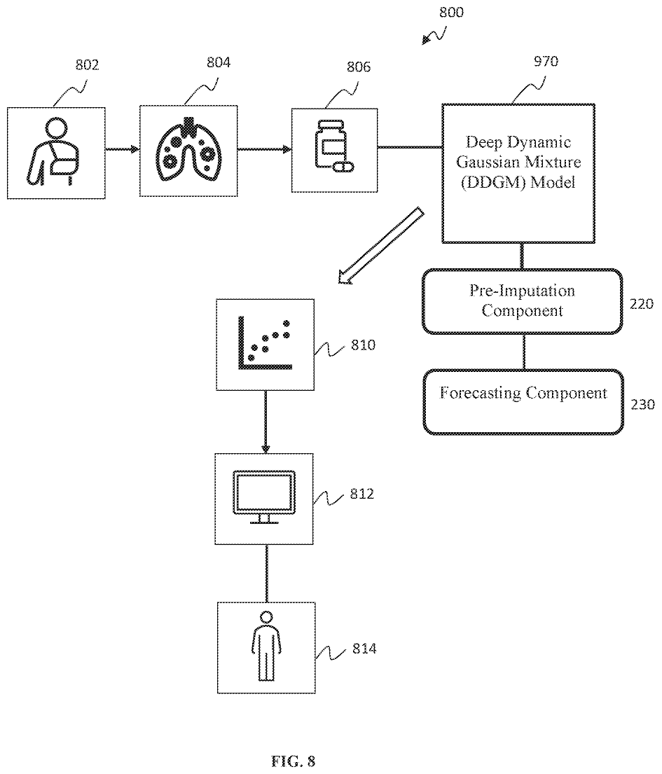

FIG. 8 is a block/flow diagram 800 of a practical application of the DDGM, in accordance with embodiments of the present invention.

In one practical example, a patient 802 needs to receive medication 806 (dialysis) for a disease 804 (kidney disease). Options are computed for indicating different levels of dosages of the medication 806 (or different testing). The exemplary methods employ the DDGM model 970 via a pre-imputation component 220 and a forecasting component 230. In one instance, DDGM 970 can chose the low-dosage option (or some testing option) for the patient 802. The results 810 (e.g., dosage or testing options) can be provided or displayed on a user interface 812 handled by a user 814.

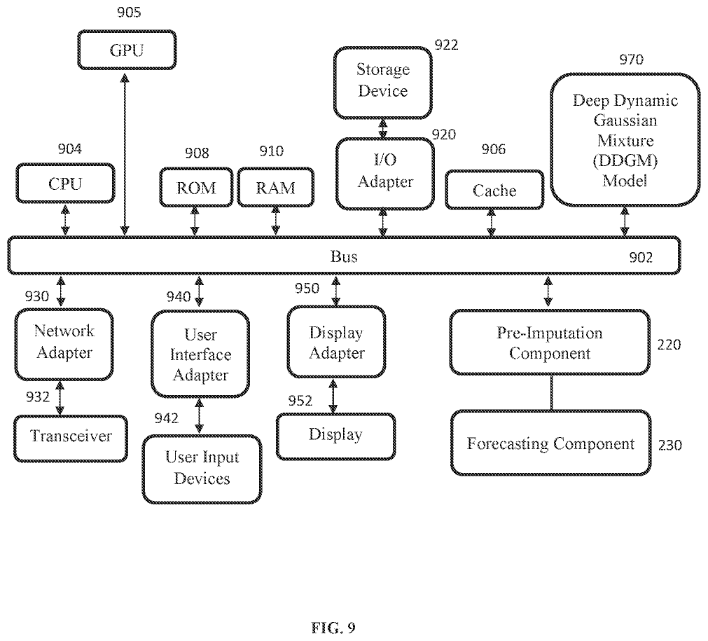

FIG. 9 is an exemplary processing system for the DDGM, in accordance with embodiments of the present invention.

The processing system includes at least one processor (CPU) 904 operatively coupled to other components via a system bus 902. A GPU 905, a cache 906, a Read Only Memory (ROM) 908, a Random Access Memory (RAM) 910, an input/output (I/O) adapter 920, a network adapter 930, a user interface adapter 940, and a display adapter 950, are operatively coupled to the system bus 902. Additionally, DDGM 970 can be employed to execute a pre-imputation component 220 and a forecasting component 230.

A storage device 922 is operatively coupled to system bus 902 by the I/O adapter 920. The storage device 922 can be any of a disk storage device (e.g., a magnetic or optical disk storage device), a solid-state magnetic device, and so forth.

A transceiver 932 is operatively coupled to system bus 902 by network adapter 930.

User input devices 942 are operatively coupled to system bus 902 by user interface adapter 940. The user input devices 942 can be any of a keyboard, a mouse, a keypad, an image capture device, a motion sensing device, a microphone, a device incorporating the functionality of at least two of the preceding devices, and so forth. Of course, other types of input devices can also be used, while maintaining the spirit of the present invention. The user input devices 942 can be the same type of user input device or different types of user input devices. The user input devices 942 are used to input and output information to and from the processing system.

A display device 952 is operatively coupled to system bus 902 by display adapter 950.

Of course, the processing system may also include other elements (not shown), as readily contemplated by one of skill in the art, as well as omit certain elements. For example, various other input devices and/or output devices can be included in the system, depending upon the particular implementation of the same, as readily understood by one of ordinary skill in the art. For example, various types of wireless and/or wired input and/or output devices can be used. Moreover, additional processors, controllers, memories, and so forth, in various configurations can also be utilized as readily appreciated by one of ordinary skill in the art. These and other variations of the processing system are readily contemplated by one of ordinary skill in the art given the teachings of the present invention provided herein.

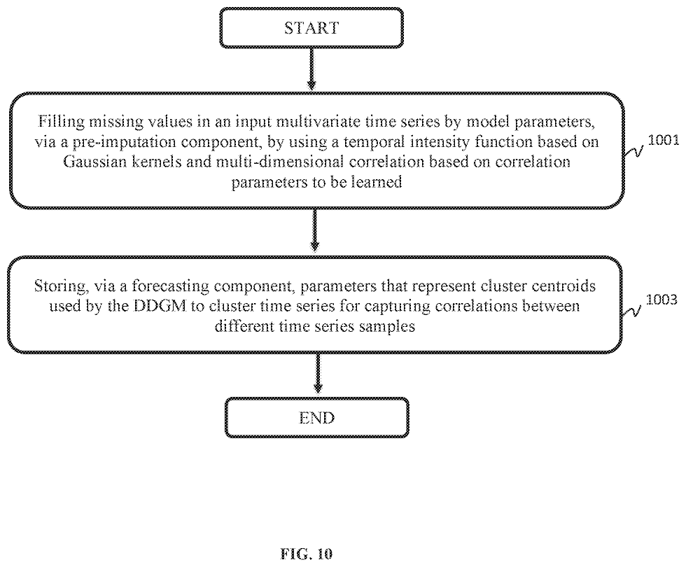

FIG. 10 is a block/flow diagram of an exemplary method for executing the MILD, in accordance with embodiments of the present invention.

At block 1001, filling missing values in an input multivariate time series by model parameters, via a pre-imputation component, by using a temporal intensity function based on Gaussian kernels and multi-dimensional correlation based on correlation parameters to be learned.

At block 1003, storing, via a forecasting component, parameters that represent cluster centroids used by the DDGM to cluster time series for capturing correlations between different time series samples.

As used herein, the terms “data,” “content,” “information” and similar terms can be used interchangeably to refer to data capable of being captured, transmitted, received, displayed and/or stored in accordance with various example embodiments. Thus, use of any such terms should not be taken to limit the spirit and scope of the disclosure. Further, where a computing device is described herein to receive data from another computing device, the data can be received directly from the another computing device or can be received indirectly via one or more intermediary computing devices, such as, for example, one or more servers, relays, routers, network access points, base stations, and/or the like. Similarly, where a computing device is described herein to send data to another computing device, the data can be sent directly to the another computing device or can be sent indirectly via one or more intermediary computing devices, such as, for example, one or more servers, relays, routers, network access points, base stations, and/or the like.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module,” “calculator,” “device,” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical data storage device, a magnetic data storage device, or any suitable combination of the foregoing. In the context of this document, a computer readable storage medium may be any tangible medium that can include, or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described below with reference to flowchart illustrations and/or block diagrams of methods, apparatus (systems) and computer program products according to embodiments of the present invention. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks or modules.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks or modules.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks or modules.

It is to be appreciated that the term “processor” as used herein is intended to include any processing device, such as, for example, one that includes a CPU (central processing unit) and/or other processing circuitry. It is also to be understood that the term “processor” may refer to more than one processing device and that various elements associated with a processing device may be shared by other processing devices.

The term “memory” as used herein is intended to include memory associated with a processor or CPU, such as, for example, RAM, ROM, a fixed memory device (e.g., hard drive), a removable memory device (e.g., diskette), flash memory, etc. Such memory may be considered a computer readable storage medium.

In addition, the phrase “input/output devices” or “I/O devices” as used herein is intended to include, for example, one or more input devices (e.g., keyboard, mouse, scanner, etc.) for entering data to the processing unit, and/or one or more output devices (e.g., speaker, display, printer, etc.) for presenting results associated with the processing unit.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the principles of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A method for managing data of dialysis patients by employing a Deep Dynamic Gaussian Mixture (DDGM) model to forecast medical time series data, the method comprising:
filling missing values in an input multivariate time series by model parameters, via a pre-imputation component, by using a temporal intensity function based on Gaussian kernels and multi-dimensional correlation based on correlation parameters to be learned; and
storing, via a forecasting component, parameters that represent cluster centroids used by the DDGM to cluster time series for capturing correlations between different time series samples.

2. The method of claim 1, wherein the temporal intensity function models temporal relationships between time steps.

3. The method of claim 2, wherein the temporal intensity function is based on an inverse distance weighting mechanism.

4. The method of claim 1, wherein the multi-dimensional correlation captures correlations between different dimensions of the input multivariate time series.

5. The method of claim 4, wherein the multi-dimensional correlation initializes a matrix parameter ρ∈ [symbol] ^{D×D}, which is a D by D continuous matrix and each entry ρᵢⱼ represents the correlation between dimension i and j.

6. The method of claim 1, wherein the forecasting component includes an inference network and a generative network.

7. The method of claim 6, wherein the inference network infers latent variables.

8. The method of claim 7, wherein the inferred latent variables are provided to the generative network to generate another copy of cluster variables.

9. The method of claim 8, wherein, after time T, the generative network uses the generated cluster variables as its own input to iteratively generate new cluster variables for time steps after T.

10. A non-transitory computer-readable storage medium comprising a computer-readable program for managing data of dialysis patients by employing a Deep Dynamic Gaussian Mixture (DDGM) model to forecast medical time series data, wherein the computer-readable program when executed on a computer causes the computer to perform the steps of:
filling missing values in an input multivariate time series by model parameters, via a pre-imputation component, by using a temporal intensity function based on Gaussian kernels and multi-dimensional correlation based on correlation parameters to be learned; and
storing, via a forecasting component, parameters that represent cluster centroids used by the DDGM to cluster time series for capturing correlations between different time series samples.

11. The non-transitory computer-readable storage medium of claim 10, wherein the temporal intensity function models temporal relationships between time steps.

12. The non-transitory computer-readable storage medium of claim 11, wherein the temporal intensity function is based on an inverse distance weighting mechanism.

13. The non-transitory computer-readable storage medium of claim 10, wherein the multi-dimensional correlation captures correlations between different dimensions of the input multivariate time series.

14. The non-transitory computer-readable storage medium of claim 13, wherein the multi-dimensional correlation initializes a matrix parameter ρΣ [symbol] ^{D×D}, which is a D by D continuous matrix and each entry pᵢⱼ represents the correlation between dimension i and j.

15. The non-transitory computer-readable storage medium of claim 10, wherein the forecasting component includes an inference network and a generative network.

16. The non-transitory computer-readable storage medium of claim 15, wherein the inference network infers latent variables.

17. The non-transitory computer-readable storage medium of claim 16, wherein the inferred latent variables are provided to the generative network to generate another copy of cluster variables.

18. The non-transitory computer-readable storage medium of claim 17, wherein, after time T, the generative network uses the generated cluster variables as its own input to iteratively generate new cluster variables for time steps after T.

19. A system for managing data of dialysis patients by employing a Deep Dynamic Gaussian Mixture (DDGM) model to forecast medical time series data, the system comprising:
a pre-imputation component for filling missing values in an input multivariate time series by model parameters by using a temporal intensity function based on Gaussian kernels and multi-dimensional correlation based on correlation parameters to be learned; and
a forecasting component for storing parameters that represent cluster centroids used by the DDGM to cluster time series for capturing correlations between different time series samples.

20. The system of claim 19, wherein the forecasting component includes an inference network and a generative network, the inference network inferring latent variables, the inferred latent variables provided to the generative network to generate another copy of cluster variables.
